(19) United States
(12) Patent Application Publication
(10) Pub. No.: US 2009/0270711 A1
(43) Pub. Date: Oct. 29, 2009

(54) PRESSURE SENSORS AND MEASUREMENT METHODS

(76) Inventors: Stacey Jarvin, Brentwood (CA); Scott Phillips, Victoria (CA)

Correspondence Address: HAMILTON IP LAW, PC, 331 W. 3RD ST., NEWVENTURES CENTER SUITE 120, DAVENPORT, IA, 52801, US

(21) Appl. No.: 12/090,068

(22) PCT Filed: Oct. 13, 2006
(86) PCT No.: PCT/CA2006/001704

§ 371 Date: Dec. 30, 2008

Related U.S. Application Data
(60) Provisional application No. 60/726,203, filed on Oct. 14, 2005.

(51) Int. Cl. A61B 3/16

(52) U.S. Cl. 600/402; 600/398

(57) ABSTRACT

A sensor for measuring pressure within anatomical structures has an impulse mechanism for delivering a mechanical impulse to the anatomical structure and a sensing mechanism for monitoring a mechanical response of the anatomical structure to the impulse. The sensor has application in measuring Intra-Ocular Pressure (IOP). The sensor may also be applied for measuring/pressures within other anatomical structures such as the heart or blood vessels. In one embodiment the impulse mechanism comprises a voice coil and the sensing mechanism comprises a piezoelectric film that generates a signal when it is distorted by motion of the anatomical structure.

### CROSS-REFERENCE TO RELATED APPLICATION

This application claims priority from U.S. patent application No. 60/726,203 filed on 14 Oct. 2005. For purposed of the United States of America, this application claims the benefit of U.S. patent application No. 60/726,203 filed on 14 Oct. 2005 under 35 U.S.C. § 119, which is hereby incorporated herein by reference.

### TECHNICAL FIELD

This invention relates to apparatus and methods for measuring pressure in anatomical structures. Embodiments of the invention have particular application to measuring intra-ocular pressure (“IOP”). Specific embodiments of the apparatus permit logging of IOP over extended periods. Other embodiments of the invention have application for measuring pressure in other anatomical structures such as chambers of the heart, blood vessels or the like.

### BACKGROUND

Excessive IOP is associated with eye diseases such as glaucoma. Devices for measuring IOP are important tools for diagnosing such diseases of the eye. Treatment of these diseases generally involves medication or surgery. Subsequent monitoring of IOP is important for assessing the success of the treatment.

Various devices exist for measuring IOP. These include:
air puff tonometers, or rebound tonometers, which provide instantaneous measurements of IOP;
Goldman and Schiotz tonometers which provide measurements of very short term averages of IOP; and,
Langham tonometers which provide measurements of short term variations in IOP.

Each of these devices relate the internal pressure of the eye to the force taken to applanate (flatten) a certain area of the cornea or the force taken to indent the cornea by a known amount. Various means are used to determine the force and the amount of indentation. Each of these devices only captures the intraocular pressure at one sitting. The pressure is known to vary substantially over a day, and variation can only be captured by numerous sittings.

Manometers can also be used to measure IOP directly by way of ocular cannulation. Manometry is undesirably invasive for routine application.

It can be desirable to obtain information regarding pressures within other anatomical structures, such as blood vessels or the heart, for example, for various reasons. Measurement of pressure in the heart and blood vessels is very useful in the diagnosis and treatment monitoring of heart disease and vessel blockages.

The inventors have identified various needs. These include:
There remains a need for practical apparatus and methods for measuring IOP. There is a particular need for such apparatus and methods which are capable of monitoring IOP over extended periods.
There is a need for effective devices and methods for monitoring ocular perfusion pressure.
There is a need for methods and apparatus for monitoring IOP over extended periods that can be practised without impeding unnecessarily a subject's vision.
There is a need for methods and apparatus for monitoring pressures within various anatomical structures such as the heart and blood vessels, for example.

### SUMMARY

Embodiments of this invention provide methods and apparatus for measuring pressure within anatomical structures. One aspect of the invention provides methods and apparatus for measuring IOP. The methods and apparatus for measuring IOP operate by applying a mechanical impulse to the eye and monitoring one or more characteristics of the mechanical response of the eye to the impulse.

Apparatus provided in another aspect of the invention comprises an impulse mechanism, a sensing mechanism and a control and processing mechanism. The impulse mechanism applies a force impulse to the eye. In response to the impulse, the material of the eye moves (i.e. has a mechanical response). The material of the eye continues to move in response to the impulse after the impulse has been delivered. The impulse is preferably of short duration compared to the natural frequency of oscillation of the eye mass. In some embodiments the impulse has a duration of less than 7 ms. In some embodiments the impulse has a duration in the range of 2-5 ms.

The sensing mechanism monitors the mechanical response of the eye to the impulse. In some embodiments, the sensing mechanism monitors motion of a surface of the eye resulting from the force impulse. Such surface motion typically has the form of a damped oscillation. Various characteristics of the damped oscillation may be measured. A value representative of IOP may be obtained based upon the measured characteristics. In some embodiments, the measured characteristics comprise a period of the oscillation.

Various impulse mechanisms and sensing mechanisms are described herein. In some embodiments, the impulse mechanism comprises a part that is in contact with the surface of the eye and can be displaced toward the eye to cause a small inward displacement of the eye surface.

The methods and apparatus may be applied to provide measurement durations and intervals appropriate to capturing pulsatile variations in the eye pressure (i.e. variations in IOP due to a patient's cardiac cycle). In some such embodiments, measurements of IOP are made at frequencies of 30 Hz or more. In some such embodiments, IOP measurements may be made at a rate on the order of 200 Hz. Embodiments of the invention may have the capability of monitoring and logging IOP over short terms (seconds) or long terms (several hours or more).

Another aspect of the invention provides apparatus and associated methods that can monitor and log IOP over extended periods.

Another aspect of the invention provides apparatus and associated methods that measure both IOP and blood pressure. An ocular perfusion pressure can be determined from the IOP and blood pressure. The apparatus may calculate the perfusion pressure.

Another aspect of the invention provides apparatus and associated methods that determine IOP based upon specific aspects of the mechanical response of an eye to an impulse force.

Other aspects of the invention provide apparatus and methods for measuring pressure within other anatomical structures such as the heart, blood vessels or the like. Such apparatus may comprise implantable sensors for monitoring such pressures over extended periods.

Further aspects of the invention and features of specific embodiments of the invention are described below.

### BRIEF DESCRIPTION OF DRAWINGS

In drawings which illustrate non-limiting embodiments of the invention:

FIG. 1 shows IOP measurement apparatus according to an embodiment of the invention that uses a voice coil to provide an impulse;

FIG. 1A shows IOP measurement apparatus according to an embodiment of the invention that uses a floating armature voice coil system to provide an impulse;

FIGS. 2A and 2B show an alternative IOP measurement apparatus that includes a piezoelectric bimorph;

FIGS. 3A and 3B show another alternative IOP measurement apparatus that includes an inflatable bladder;

FIGS. 3C and 3D show another alternative IOP measurement apparatus that includes an inflatable bladder;

FIG. 4 shows response waveform features that may be extracted and applied in an IOP measurement method;

FIG. 5 shows an example IOP measurement device;

FIG. 6 shows another example IOP measurement device;

FIG. 7 illustrates application of the device of FIG. 6;

FIG. 8 shows a possible construction for the connecting cable of the device of FIG. 6;

FIG. 9 shows internal electronic components of an example IOP measurement apparatus;

FIG. 10 shows a device having a wireless data communication pathway; and,

FIG. 11 shows apparatus for measuring pressure within a blood vessel.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

It is desirable to provide tools for making IOP measurements that are adapted for use in various clinical applications. IOP measurement apparatus according to one or more aspects of the invention may be designed to perform in various settings. For example, embodiments of the invention may have form factors and operator interfaces that make them particularly suited for clinical settings such as:
conducting IOP screening in ophthalmologists' offices;
monitoring and logging variations in IOP over extended periods;
and the like.

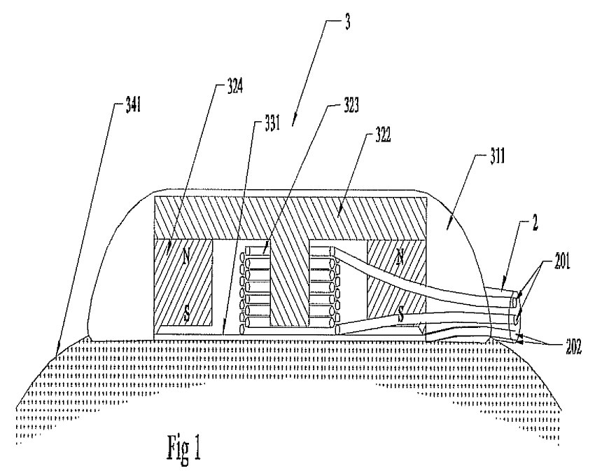

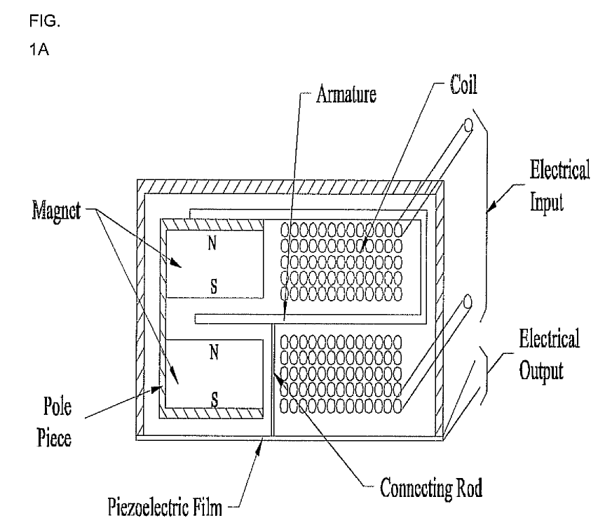

FIG. 1 shows a combined impulse mechanism and sensing mechanism (which will be referred to as a sensor 3) according to one embodiment of the invention. Sensor 3 has a voice-coil construction. A coil 323 of wire is mounted to a diaphragm 331. Coil 323 is located in a radial magnetic field. In the illustrated embodiment, the magnetic field is between a magnet 324 and a pole piece 322. Sensor 3 can be caused to apply an impulse to the surface of an eye 341 by passing a pulse of electrical current through coil 323. In the embodiment of FIG. 1, the electrical current is delivered from an external source by way of wires 201. An axial force on coil 323 results. The axial force displaces at least a central portion of diaphragm 331. Diaphragm 331 may be elastic. Diaphragm 331, in turn, applies an impulse force to the surface of eye 341.

As a result of the impulse, the material of eye 341 will move. Typically, the movement of the surface of eye 341 is in the form of a damped oscillation that continues after the exciting impulse is finished.

In the illustrated embodiment, motion of the surface of eye 341 is monitored by monitoring displacements of diaphragm 331. The sensing mechanism (diaphragm 331 in the illustrated embodiment) has a compliance that is comparable to or greater than a compliance of the eye surface so that the sensing mechanism can readily follow motions of the eye surface.

The displacement of diaphragm 331 may be monitored in various ways. In the illustrated embodiment, diaphragm 331 comprises a piezoelectric film. Such a film has the property of generating a voltage when distorted. The piezoelectric film is metallized on both sides.

Sensing wires 202 carry a signal from diaphragm 331 to a measuring circuit (not shown in FIG. 1). The measuring circuit may comprise suitable analog and/or digital filters, amplifiers, and other signal conditioning elements as is known to those skilled in the art. This measuring circuit senses and captures and/or analyzes the signal from diaphragm 331.

Sensor 3 is preferably constructed so that diaphragm 331 is stretched. When a piezoelectric film is configured as a taut diaphragm, and the diaphragm is distorted, the piezoelectric effect is mechanically magnified, and a comparatively large voltage is generated.

A sensor 3 as illustrated in FIG. 1 can provides the advantage that a reasonably large impulse can be generated efficiently by passing an electrical current through coil 323 and a diaphragm 331 comprising a piezoelectric film can detect resulting motions of the eye with great sensitivity.

FIG. 1A shows a sensor 3A having an impulse mechanism of an alternative construction. The construction shown in FIG. 1A is known as a “floating armature receiver”. An example of a floating armature receiver is described in U.S. Pat. No. 6,654,477. A floating armature receiver can provide a faster transient response than an equivalent standard voice coil because the coil does not need to be attached to the moving member. Therefore, the moving member can be lighter and capable of responding more quickly to transient motions.

In sensor 3 (or 3A) coil 323 may be used in place of a piezoelectric diaphragm to sense motion of the eye. A coil moving in a magnetic field will generate a voltage. If, immediately after the excitation impulse has ceased, the coil is connected to an input of a suitable amplifier, the output of the amplifier will represent the motion of the eye surface. Using coil 323 to sense motion of the eye surface has the disadvantage that it is typically not practical to sense motion of the eye surface while the impulse is being delivered. On the other hand, the simplicity of using coil 323 for the dual purposes of delivering an impulse and detecting a signal that indicates motion of the eye may be preferable in some circumstances.

In the further alternative, two coils may be provided. A coil 323 may be part of an impulse mechanism while a separate coil (not shown) may be provided as part of a sensing mechanism.

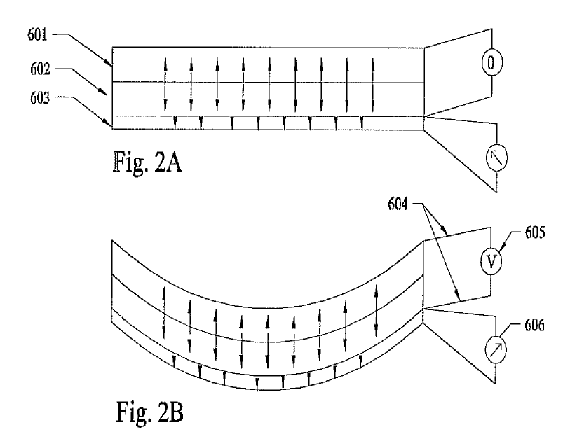

FIGS. 2A and 2B show a sensor 3B having an alternative arrangement. Sensor 3B includes a piezoelectric actuator for delivering an impulse to an eye. In the illustrated embodiment, the actuator comprises two piezoelectric elements 601 and 602. Elements 601 and 602 are polarized oppositely as shown, by way of example, by the arrows. Each of elements 601 and 602 has electrically conducting surfaces. Elements 601 and 602 are bonded together in a bimorph arrangement.

As shown in FIG. 2B, elements 601 and 602 bend when a suitable input voltage 605 is applied to wires 604. Application of input voltage 605 causes elements 601 and 602 to expand in opposite directions. In FIG. 2B the degree of bending of the structure is exaggerated significantly for illustrative purposes. By applying a pulse of voltage 605 while sensor 3B is held against a person's eye, an impulse may be delivered to the eye.

In the embodiment of FIGS. 2A and 2B, motion of the eye is sensed by a piezoelectric film 603. Film 603 is in front of, and may be bonded to the bimorph structure made up of elements 601 and 602. In some embodiments, film 603 is polarized in the same way as the adjacent element 602 as shown by the arrowheads in FIGS. 2A and 2B. Film 603 generates an output signal in response to bending. Where film 603 is bonded to bimorph 601, 602, the output signal is generated whenever bimorph 601, 602 bends.

FIG. 2A shows sensor 3B in a relaxed position with no voltage applied, and no voltage output from film 603. FIG. 2B shows sensor 3B when stimulating voltage 605 is applied to cause bimorph 601, 602 to flex and a resulting voltage signal 606 is produced as a result of the distortion of film 603.

As an alternative to the construction shown in FIGS. 2A and 2B, one of elements 601 and 602 may be a non piezoelectric material, such as metal. The resulting bimorph may have reduced sensitivity. Other possible variations in sensor 3B include:
providing elements 601 and 602 with alternative piezoelectric polarizations;
providing configurations that have different electrical voltage connections.

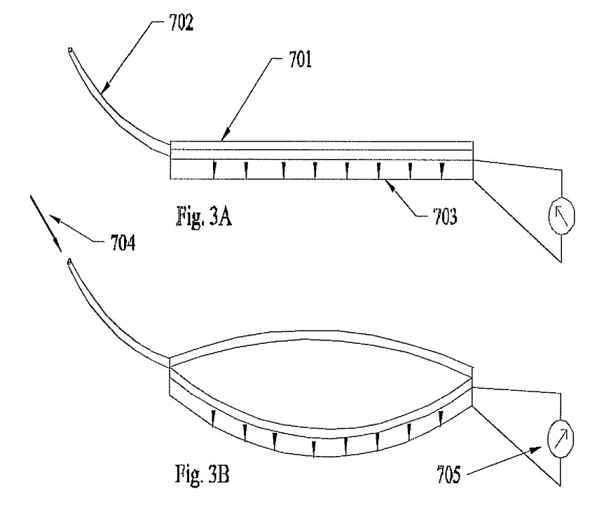

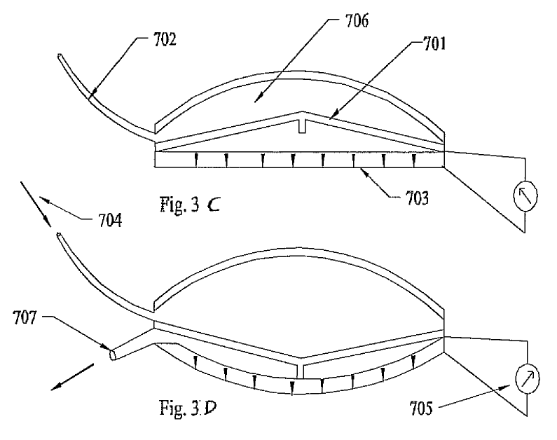

FIGS. 3A and 3B show schematically a sensor 3C in which an impulse may be delivered to an eye by introducing a pulse of a fluid into a bladder. Sensor 3C has a flat flexible bladder 701. Bladder 701 is fed air by a tube 702 from a source of air pressure 704. A momentary pulse of air from source 704 into bladder 701 will produce a momentary inflation of bladder 701 as shown (greatly exaggerated for clarity) in FIG. 3B. Sudden inflation of bladder 701 will apply a momentary force to the surface of the eye.

Bladder 701 is laminated to a piezoelectric film 703. Film 703 senses motion of the eye. In the embodiment of FIGS. 3A and 3B, flexing of film 703 produces a signal 705 that is related to eye surface displacement.

FIGS. 3C and 3D show a sensor 3D which is similar to sensor 3C in that it is powered by a pressurized gas. In sensor 3D, a pressurized gas (e.g. compressed air) is introduced via tube 702 into a chamber 706. As more gas is introduced into chamber 706, the pressure within chamber 706 increases. The pressure exerts a force on a flexible diaphragm 708. Eventually the pressure on diaphragm 708 causes it to flex “over center”. The flexing allows a diaphragm 708 to rapidly impinge on piezoelectric film 703. This delivers an impulse to an eye that is adjacent to film 703.

In the illustrated embodiment, the impulse is delivered by a member 709 that projects from diaphragm 708. A port 707 is provided to release air from the space between diaphragm 708 and film 703.

Diaphragm 708 may be returned to its original position, for example, by one or more of:
reducing the pressure in chamber 706;
designing diaphragm 708 in a manner that causes diaphragm 708 to return to the original configuration of FIG. 3C;
providing a separate bias mechanism to return diaphragm 708 to its original configuration; or
the like.

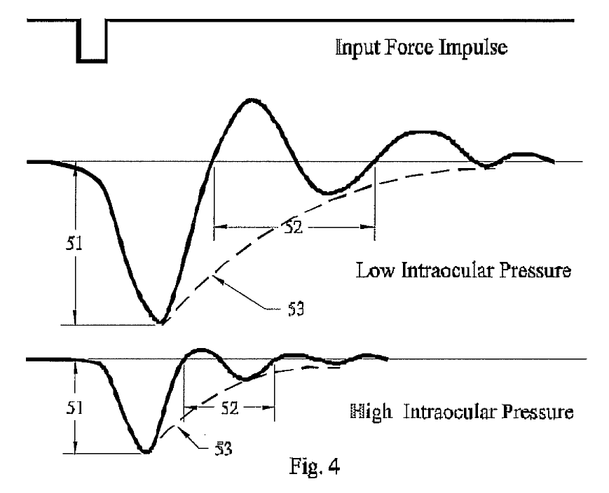

Once a signal that represents motion of the eye has been obtained, the signal can be processed to obtain a value or values representative of IOP. FIG. 4 shows example time-varying signals that may be produced when a method according to the invention is used to study eyes having lower (upper graph) and higher (lower graph) IOP.

Various features of such time-varying signals may be measured. These include:
Initial displacement, parameter 51, is seen to be related to IOP. Lower values for IOP correlate to higher values for initial displacement 51.
The natural period 52 of oscillation of the eye is seen to be related to IOP. Period 52 tends to be shorter (higher frequency) for higher values of IOP.
A parameter, such as a decay constant, or more complicated function representing the manner in which a signal decays, shown schematically by the dashed line 53, is related to IOP. The signal decays more quickly for higher values of IOP.

These features can be made visible to the eye by observing the signal using an electronic measuring instrument such as an oscilloscope. In preferred embodiments of the invention, one or more of these features is automatically extracted from the signal, for example by suitable digital processing.

In some embodiments of the invention, the signal is captured, digitized, and the resulting digital signal data is analyzed to obtain the signal features by any suitable digital signal processing methods and/or apparatus. A wide range of such methods and apparatus is known to those skilled in the art. A value representative of IOP may then be obtained by computing a function of one or more of the features. In some embodiments, a lookup table or calibration function is provided to obtain IOP values from the extracted value(s) or a function thereof.

The relationship of the extracted features to IOP may be empirically calibrated for any individual sensor and processing combination. Apparatus according to the invention may produce an output that represents IOP on an absolute scale (such as mmHg) or may be used without (or with) calibration to an absolute scale to indicate variations in the IOP of a particular eye in cases where absolute measurements of IOP are not needed.

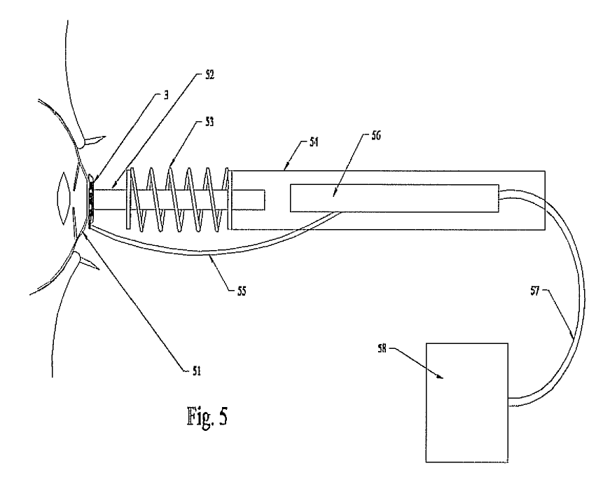

Various types of instrument may incorporate sensors of the type described above for the purpose of measuring IOP. Some examples are described below. FIG. 5 shows an IOP measurement device 50 having a sensor 3 mounted on the end of a probe. Sensor 3 may have any of the constructions described above or may combine features of the sensors described above. Other sensors capable of measuring motion of the eye in response to an impulse may also be used.

Device 50 may be hand-held or preferably mounted on a stand for the purpose of taking momentary or short term readings of IOP. In operation, sensor 3 is pressed against the cornea 51 of an eye. Sensor 3 is mounted on a rod 52 and is pressed against the eye by a force, provided by way of example by a spring 53. Rod 52 and spring 53 are mounted to a support 54 which may be hand-held or affixed to a stand.

Signal conductors 55 carry signals from sensor 3 to control and processing electronics 56 and also carry from electronics 56 to sensor 3 signals to trigger or cause sensor 3 to deliver an impulse to the eye. Optionally, electronics 56 are connected to a host computer, network, additional electronic control mechanism or the like. Such a connection may be implemented by way of any suitable wired or wireless communication technology.

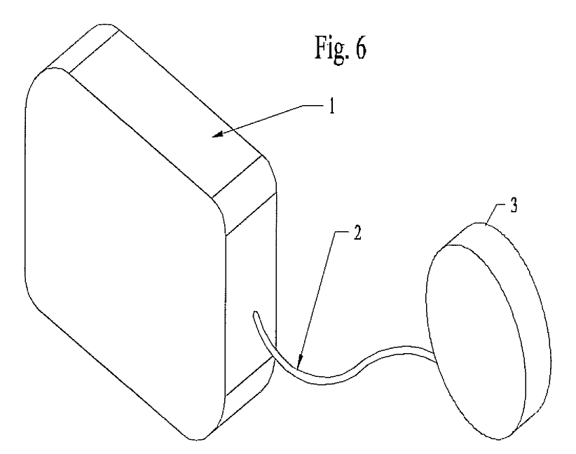

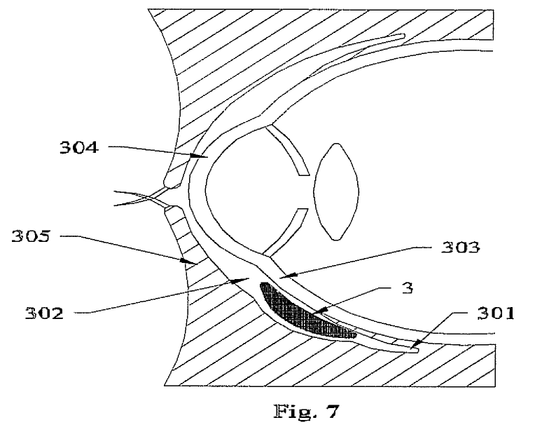

FIG. 6 shows a device 60 in which sensor 3 is small and encapsulated so that it can be inserted to bear against the sclera of the eye as shown in FIG. 7. Sensor 3 may be retained in a desired position against sclera 303 by providing a thin ring around the raised cornea (as disclosed U.S. Pat. No. 4,089,329). Sensor 3 is held against the eyeball by the pressure of eyelid 305, aided by the surface tension of the eye fluids. Preferably sensor 3 is located in such a position that it does not impair normal vision. Any apparatus for supporting sensor 3 in place preferably also does not obstruct normal vision. Providing an IOP monitoring system that does not obstruct normal vision facilitates patient acceptance of monitoring over longer periods.

Alternatively, sensor 3 may be affixed to the eyeball by fastening it to the sclera with a removable adhesive specially suited for the purpose.

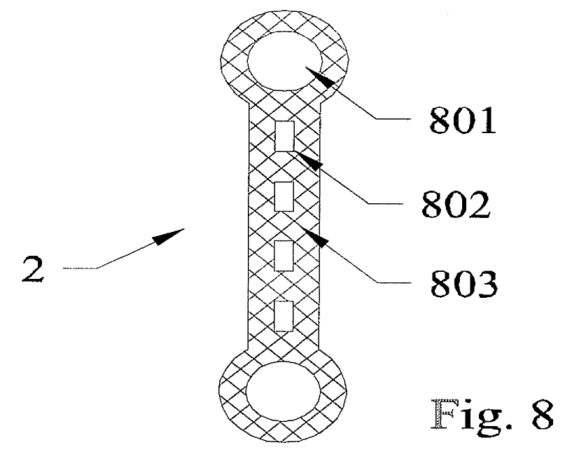

FIG. 8 shows a cross section of the connecting portion 2 of the device of FIG. 6. This connecting portion carries signals between sensor 3 of device 60 and control portion 1 of device 60. Connecting portion 2 may include several electrically conductive tracks 802 laminated between layers of plastic film 803. Connecting portion 2 is preferably flattened, to provide less obstruction to the eye opening.

Although it is not preferred, connecting portion 2 could comprise a number of metallic insulated wires in a bundle. Connecting portion 2 may also contain tubes 802 to conduct air impulses in the case that compressed air is used directly or indirectly to deliver an impulse to the eye (as, for example, shown in FIGS. 3A to 3D) and/or to convey hydration or anesthetic fluids to the eye.

As shown in FIG. 6, the control portion 1 of device 60 is located external to the eye. Control portion 1 may be temporarily affixed to the head, a headband or spectacles or located elsewhere on the body. Control portion 1 contains the electronic and mechanical devices necessary to drive sensor 3 and to process signals from sensor 3. Control portion 1 may comprise a data logger that stores on a suitable data storage medium a record of signals from sensor 3, features extracted from such signals, or values computed from such features. The data logger may record values correlated to IOP periodically over a period long enough to monitor for variations in IOP. For example, the data logger may record such values at periods spaced apart over 24 hours to obtain a record which can be used to evaluate diurnal and nocturnal variations in IOP.

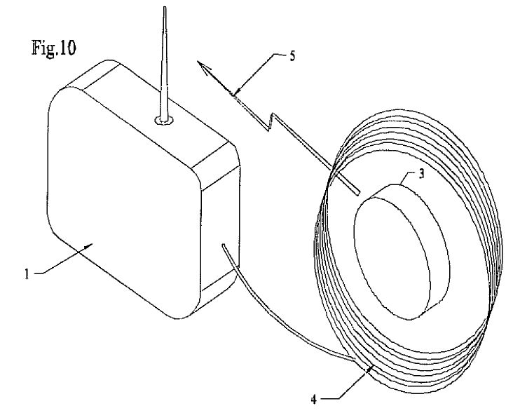

FIG. 10 shows a device 100 that is similar to device 60 except that sensor 3 and control portion 1 are in wireless communication with one another. Preferably, power and control signals for sensor 3 are provided remotely from control portion 1. Electrical power may be provided to sensor 3 by way of AC induction. In the illustrated embodiment, a pickup coil in sensor 3 collects energy from a remotely mounted transmitting coil 4. Sensed output information from sensor 3 is shown schematically as being returned by radio communication 5. Suitable electromagnetic communication methods and variations thereon are well known to those skilled in the art and are therefore not described in detail herein.

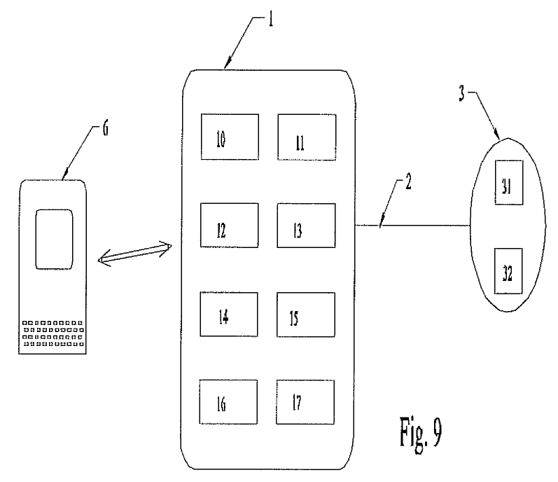

FIG. 9 is a block diagram of a device 90 according to an embodiment of the invention. Device 90 may have a form factor like that of any of devices 50, 60 or 100 for example. The components of device 90 may be implemented in software, in hardware or as combinations of hardware and software.

Device 90 includes a timing control 10 that provides basic timekeeping functions for recordkeeping and scheduling operations. A sensor driver 11 provides excitation energy for sensor 3. The excitation energy is provided in a form suitable for the type of impulse mechanism provided by sensor 3. For example:
Where the impulse mechanism comprises a current coil, sensor driver 11 provides an electrical current impulse.
Where the impulse mechanism comprises a piezoelectric actuator, such as a piezoelectric bimorph, sensor driver 11 provides a voltage impulse.
Where the impulse mechanism is operated by a fluid, such as air, sensor driver 11 provides a pulse of air or other fluid.

Sensor signal conditioning means 13 provides electrical circuits which receive, isolate and amplify signals from sensor 3 whether those signals comprise output from a piezoelectric film sensor, a coil, or some other sensor that detects the mechanical response of the eye to an impulse.

Signal processing means 12 extracts features of interest from signals conditioned by signal conditioning means 13. For example, signal processing means 12 may extract one or more of peak amplitude 51, decay time constant 53 and ringing frequency 52 (See FIG. 4). In some embodiments, signal processing means 12 comprises a digitizer (e.g. an analog-to-digital converter (“ADC”)) that captures values of the time-variable signals and at least temporarily stores the signal values for analysis. This analysis may include analog and digital signal processing involving mathematical algorithms to extract the features of the signal.

Signal processing means 12 may convert the extracted features to IOP values by executing an internal computer conversion algorithm. The algorithm may include parameters determined as a result of design or factory calibration. Such parameters may be encoded in software or firmware, stored on a memory associated with device 90 or otherwise preserved for use by device 90. In some embodiments, the parameters may be checked or modified by comparison to reference IOP measurements, as desired.

Power source 15 supplies power to other parts of device 90. Power source 15 typically comprises a battery or line power supply. However, any suitable power source may be used.

Data store 14 stores software, if required for operation of device 90, any calibration constants, information defining a schedule of IOP measurements to perform and all data generated. Data store 14 may comprise non-volatile semiconductor memory but any other suitable data storage medium may be used. Data store 14 may comprise different parts for storing different information. Data store 14 may optionally include a removable part what may be used to transfer data from control portion 1 to some other device.

Communication means 16 allows stored data to be transferred to a host computer system or to paper records. Communication means 16 may comprise, without limitation, removable or permanently connected wiring (electrical or optical), radio communication of any suitable frequency or frequencies and protocol, or optical communication of any suitable wavelength or wavelengths and protocol.

Computer 6 may comprise any suitable device including a personal computer, a networked mainframe computer, a network server, a network data store, a portable digital device such as a personal digital assistant (“PDA”) or a custom device. Computer 6 may run analytical and display software configured for the purpose, or it may involve custom software with specialized functions related to this device.

Fluid delivery system 17 includes components such as reservoirs, pumps and metering devices to deliver fluids (which may include anesthetics or other drugs, hydrating solutions, or the like) to the vicinity of the eye. Timing control 10 may cause fluid delivery system to operate periodically to deliver a small quantity of fluid to the vicinity of the eye.

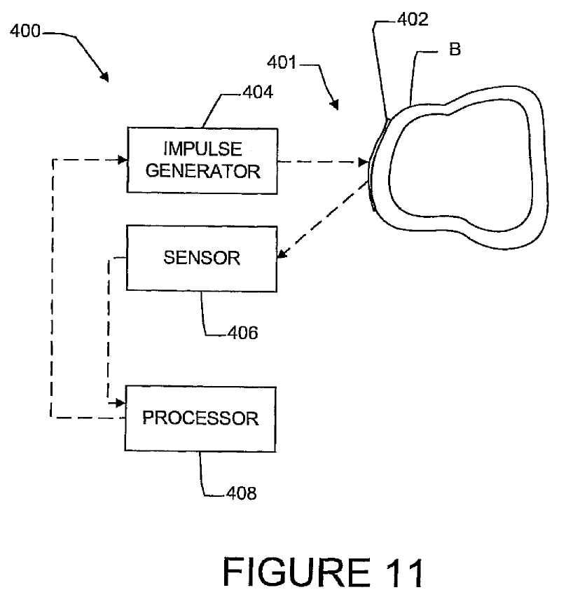

FIG. 11 shows pressure sensor apparatus 400 that is adapted for measuring pressure in a blood vessel B. Apparatus 400 comprises a transducer 401 comprising a flexible diaphragm 402 that sits against an outer surface of blood vessel B, an impulse generator 404 configured to deliver a physical impulse to diaphragm 402 and blood vessel B, and a sensor 406 that detects motion of diaphragm 402. Apparatus 400 also comprises a processor 408 that: evaluates a characteristic of the motion of diaphragm 402 after the delivery of an impulse; and based at least in part on that characteristic determines a corresponding pressure within blood vessel B. Processor 408 may also control the operation of impulse generator 404.

In some embodiments, transducer 401 is implanted surgically adjacent to blood vessel B or another anatomical structure for which it is desirable to monitor an internal pressure. In such embodiments, transducer 401 may be coated in a suitable bio-compatible material. In such embodiments, processor 408 may comprise a part that is implanted within a subject's body and another external part that communicate with one another by way of suitable telemetry. In some embodiments, transducer 401 is coupled to provide feedback to another implanted device such as a heart pacemaker or the like.

Diaphragm 402 may be formed to have an undistorted shape that conforms generally to the shape of an anatomical structure, such as a blood vessel B for which the pressure is to be monitored. For example, diaphragm 402 may be pre-curved to have a cylindrical or spherical conformation when it is undistorted.

In some specific embodiments of transducer 401 impulse generator 404 may comprise a voice-coil as shown in FIGS. 1 and 1A, a piezoelectric bimorph as shown in FIGS. 2A and 2B, or an inflatable bladder as shown in FIGS. 3A and 3B. In some specific embodiments of transducer 401, sensor 406 comprises a piezoelectric film, as shown in FIG. 1, 2A or 2B or a coil, such as a floating armature voice coil, as shown in FIG. 1A.

In one embodiment, impulse generator 404 comprises a voice coil coupled to deliver a mechanical impulse to diaphragm 402 and sensor 406 comprises a piezoelectric element coupled to diaphragm 402 so that the piezoelectric element is distorted by motion of diaphragm 402. The piezoelectric element may comprise a piezoelectric film disposed on a surface of diaphragm 402 or disposed in a layer within diaphragm 402.

Certain implementations of the invention comprise data processors which execute software instructions which cause the processors to perform a method of the invention. For example, control portion 1 of device 90 may comprise a programmed computer executing software that includes components that cause the programmed computer to implement the functions of one or more of: timing control 10, parts of sensor signal conditioning means 13, part or all of signal processing means 12; and/or coordination of the overall operation of device 90. The invention may also be provided in the form of a program product. The program product may comprise any medium which carries a set of computer-readable signals comprising instructions which, when executed by a data processor, cause the data processor to execute a method of the invention. The program product may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

While a number of exemplary aspects and embodiments have been discussed above, those of skill in the art will recognize certain modifications, permutations, additions and sub-combinations thereof. For example:
Some data processing may be performed in sensor 3. Data may be stored in a memory located on or in sensor 3. Control circuits to trigger an impulse and the subsequent acquisition of information about eye motion may reside in sensor 3. Given a suitably compact source of energy, an entire device for monitoring IOP may be provided in the form of a sensor 3 small enough to be received between the eye and the outer part of the eyelid.
Where it is desired that apparatus automatically determine perfusion pressure, the apparatus may incorporate a blood pressure monitor of any suitable type or may have an interface to receive blood-pressure values from external blood-pressure measuring instruments. The interface may comprise a manual input to accept manually-input blood pressure values but preferably receives blood pressure signals directly. The interface may include a mechanism whereby a blood-pressure measurement can be triggered to be taken at substantially the same time as the apparatus takes an IOP measurement. Another aspect of the invention provides apparatus and associated methods that measure both IOP and blood pressure. The blood pressure may comprise systolic, diastolic and/or mean blood pressure values. Apparatus may compute a perfusion pressure from the IOP and blood pressure.

It is therefore to be understood that the invention has a wide range of aspects. Any claims hereafter introduced should be interpreted to include all such modifications, permutations, additions and sub-combinations as are within their true spirit and scope.

1. A sensor for use in the measurement of pressure within an anatomical structure, the sensor comprising an impulse mechanism for delivering a mechanical impulse to the anatomical structure and a sensing mechanism for monitoring a mechanical response of the anatomical structure to the impulse.

2. A sensor according to claim 1 wherein the impulse mechanism comprises a surface to be placed in contact with the anatomical structure and a mechanism for applying an impulse to the surface.

3. A sensor according to claim 2 wherein the surface comprises a surface of a flexible diaphragm.

4. A sensor according to claim 3 wherein the diaphragm is elastic.

5. A sensor according to claim 3 wherein the diaphragm is maintained under tension.

6. A sensor according to claim 5 wherein the diaphragm is formed to have a configuration substantially conforming to a cylindrical surface when undistorted.

7. A sensor according to claim 5 wherein the diaphragm is formed to have a configuration substantially conforming to a spherical surface when undistorted.

8. A sensor according to claim 1 wherein the sensing mechanism comprises a piezoelectric film.

9. A sensor according to claim 1 wherein the sensing mechanism comprises a coil movable in relation to a magnetic field.

10. A sensor according to claim 1 wherein the impulse mechanism comprises a coil disposed in a magnetic field.

11. A sensor according to claim 1 wherein the impulse mechanism comprises a bladder and a conduit connected to deliver a fluid to the bladder.

12. A sensor according to claim 1 wherein the impulse mechanism comprises a piezoelectric bimorph.

13. A sensor according to claim 1 wherein the sensing mechanism is configured to be placed against a human eye for measurement of intraocular pressure.

14. A sensor according to claim 13 comprising a thin ring on the sensor.

15. A sensor according to claim 13 wherein the sensing mechanism has a compliance that is at least comparable to or greater than a typical compliance of the eye.

16. A sensor according to claim 13 in combination with a processing system connected to receive a signal from the sensing mechanism and to determine a pressure within the anatomical structure based at least in part on the signal.

17. A sensor combination according to claim 16 wherein the processing system is adapted to extract from the signal at least one feature and to compute a pressure value based at least in part on the at least one feature.

18. A sensor combination according to claim 17 wherein the mechanical response comprises a surface motion of the anatomical structure.

19. A sensor combination according to claim 18 wherein the processing system is adapted to extract from the signal a feature representing an amplitude of the surface motion of the anatomical structure.

20. A sensor combination according claim 18 wherein the processing system is adapted to extract from the signal a feature representing a characteristic of a decay of the surface motion of the anatomical structure.

21. A sensor combination according to claim 20 wherein the characteristic of the decay comprises a decay time constant.

22. A sensor combination according to claim 18 wherein the processing system is adapted to extract from the signal a feature representing a frequency or period of the surface motion of the anatomical structure.

23. A sensor combination according to claim 16 wherein the processing system is configured to compute an IOP value based at least in part on one or more of an initial amplitude, a vibration frequency and a decay time constant of eye motion resulting from a step impulse of applied force.

24. A sensor combination according to claim 16 wherein the anatomical structure is an eye and the sensor combination comprises a control system configured to cause the impulse mechanism to deliver impulses to the eye at a rate sufficient to observe pulsatile pressure changes.

25. A sensor combination according to claim 24 wherein the control system is configured to cause the device to deliver impulses to the eye at a rate in excess of any of 1 Hz; 5 Hz; 10 Hz; 15 Hz; 25 Hz and 100 Hz.

26. A sensor combination according to claim 16 wherein the sensor and processing system are connected by a connecting link comprising a fluid-delivery tube.

27. A sensor combination according to claim 16 wherein the sensor and processing system are connected by a connecting link comprising a plurality of electrical conductors encased in a flat film.

28. A sensor combination according to claim 16 comprising a mechanism for periodically delivering a fluid to a vicinity of the sensor.

29. A sensor according to claim 1 wherein the impulse mechanism comprises a coil suspended in a magnetic field and the sensor mechanism comprises a piezoelectric film.

30. A method for measuring intraocular pressure, the method comprising:
delivering an impulse to an eye;
monitoring a time-varying mechanical response of the eye to the impulse over a period of time;
computing an IOP value based upon at least one feature of the time-varying mechanical response.

31. A method according to claim 30 wherein the at least one feature comprises an initial amplitude of the mechanical response.

32. A method according to claim 30 wherein the at least one feature comprises a frequency or period of the mechanical response.

33. A method according to claim 30 wherein the at least one feature comprises a characteristic of a decay of the mechanical response.

34. A method according to claim 33 wherein the at least one feature comprises a time constant of the decay of the mechanical response.

35. A method according to claim 30 comprising repeating the method at a rate sufficient to observe pulsatile pressure changes.

36. A method according to claim 30 comprising repeating the method at a rate in excess of any of 1 Hz; 5 Hz; 10 Hz; 15 Hz; 25 Hz and 100 Hz.

37. A method according to claim 30 comprising placing a sensor in contact with the sclera of the eye under or partly under the eyelid.

38. A method according to claim 30 repeated at spaced-apart times over a period in excess of one or more of: 7 hours; 12 hours and 24 hours.

39. A method according to claim 30 comprising automatically supplying hydration to the eye.

40. A method according to claim 30 comprising automatically supplying anaesthetic to the eye.

41. A method according to claim 30 wherein monitoring the time-varying mechanical response of the eye is performed with a sensor held against the eye at least in part by surface tension.

42. A method according to claim 30 wherein monitoring the time-varying mechanical response of the eye is performed with a thin sensor held against the eye at least in part by pressure of an eyelid.

43. A method according to claim 42 wherein the sensor is located in such a position that it does not impair normal vision of the eye.

44. A method for measuring pressure within an anatomical structure the method comprising:
delivering a mechanical impulse to the anatomical structure
monitoring a time-varying mechanical response of the anatomical structure to the impulse over a period of time; and,
computing a pressure value based upon at least one feature of the time-varying mechanical response.

45. A method according to claim 44 wherein the at least one feature comprises an initial amplitude of the mechanical response.

46. A method according to claim 44 wherein the at least one feature comprises a frequency or period of the mechanical response.

47. A method according to claim 44 wherein the at least one feature comprises a characteristic of a decay of the mechanical response.

48. A method according to claim 47 wherein the at least one feature comprises a time constant of the decay of the mechanical response.

49-51. (canceled)
